(19) United States
(12) Patent Application Publication
(10) Pub. No.: US 2021/0370034 A1
(43) Pub. Date: Dec. 2, 2021

(54) Decontamination Station

(71) Applicants: Craig Crippen, Aberdeen, MD (US); LaTonya Crippen, Aberdeen, MD (US)

(72) Inventors: Craig Crippen, Aberdeen, MD (US); LaTonya Crippen, Aberdeen, MD (US)

(21) Appl. No.: 16/901,222

(22) Filed: Jun. 15, 2020

Related U.S. Application Data
(60) Provisional application No. 63/030,403, filed on May 27, 2020.

(51) Int. Cl. A61M 35/00; A61K 31/045

(52) U.S. Cl. CPC A61M 35/20; A61K 31/045

(57) ABSTRACT

A decontamination apparatus for misting a user with a decontaminating alcohol mist. The decontamination apparatus is configured to decontaminate a person and their clothing simultaneously. A frame component defines a decontamination area for the user to enter. A fluid reservoir retains a decontamination solution until it is dispensed via a plurality of nozzles. The plurality of nozzles may be positioned around the frame component, be integrated into a dispensing component that rotates around the user or be integrated into a dispensing component that partially surrounds the user and moves up and down for full coverage. A plastic or nylon chamber may be attached to the frame component to surround the decontamination area to prevent overspray.

### CROSS-REFERENCE TO RELATED APPLICATION

The present application claims priority to, and the benefit of, U.S. Provisional Application No. 63/030,403, which was filed on May 27, 2020 and is incorporated herein by reference in its entirety.

### BACKGROUND

The present invention generally relates to a decontamination system, and more specifically to an in-home personal decontamination and sanitizing station. Accordingly, the present specification makes specific reference thereto. However, it is to be appreciated that aspects of the present invention are also equally amenable to other like applications, devices and methods of manufacture.

Germs, viruses, bacteria, microbes, and other pathogens are tiny living things found everywhere in nature and are too small to be seen by the naked eye. These organisms are constantly present and persist on a wide variety of surfaces including clothing and human skin. A pathogen is a micro-organism that has the potential to cause disease. Inhaling these pathogens or introducing them into mucus membranes that exist in the eyes, nose, and mouth can lead to serious illness or even death. People carry these pathogens into their homes unknowingly because they lack a proper means to sanitize or disinfect their persons before entering their house. This puts other members of the house at greater risk of infection as they interact with the person introducing the pathogens into the house.

Viruses are by far the most common cause of infectious diseases acquired in the indoor environment in hospitals, schools, households, and other indoor environments causing considerable impact on human health. Transmission of enteric and respiratory viruses is assumed to occur predominantly directly from person to person, followed by indirect transmission through contaminated surfaces including clothing and human skin. Cleaning and disinfection of contaminated surfaces are one of the frequently implemented measures to control transmission of pathogens in indoor environments.

COVID-19 is a virus caused by a coronavirus called SARS-CoV-2. Older adults and people who have severe underlying medical conditions like heart or lung disease or diabetes seem to be at higher risk for developing more serious complications from COVID-19 illness. It is thought to spread mainly from person to person, mainly through respiratory droplets produced when an infected person coughs or sneezes. These droplets can land in the mouths or noses of people who are nearby or possibly be inhaled into the lungs. Spread is more likely when people are in close contact with one another. The spread of COVID-19 can also occur by touching a surface or object that has the virus on it and then touching one's own mouth, nose, or possibly their eyes.

Disinfection, sanitization, and sterilization are all processes of decontamination. However, these processes differ in application and effect. Sanitizers reduce the overall number of the microorganisms that make us sick on an inanimate or living surface. Sanitizers have a more broad and immediate use against a wide range of microbes to reduce the overall number of microbes on a surface, thus reducing the chances of illness. A disinfectant has a stronger effect than a sanitizer on certain pathogenic microbes. Disinfectants normally have a more focused and specific use against certain bacteria, viruses, and disease. Disinfection eliminates most pathogens but not necessarily all types of microbes. Disinfectants also require a “dwell time” to be effective, meaning they must remain on an inanimate surface for a certain period of time to be effective against the advertised microbe(s). Additionally, disinfectants are effective on inanimate surfaces. However, many disinfectants are weakened by organic matter such as living tissue such as skin. As such, disinfectants can be stronger than sanitizers, but require a more specific and meticulous application to be effective. Finally, sterilization utilizes chemical application to decontaminate, but additionally incorporates heat and/or pressure to completely eliminate any and all pathogenic microbes. Methods used in sterilization procedures include heat, ethylene oxide gas, hydrogen peroxide gas, plasma, ozone, and radiation.

Decontamination is the process that removes or destroys contamination so that infectious agents or other contaminants cannot reach a susceptible site in sufficient quantities to initiate infection, or other harmful response. Decontamination renders a person, item or material safe to handle. The level of microbial contamination is reduced enough that it can be reasonably assumed free of risk of infection transmission.

Decontamination and sanitizing agents include soap and water, hypochlorite (bleach), hydrogen peroxide, and alcohol, among others. When decontaminating human skin and clothing, the choices are more limited. Soap and water would be effective but require the user to get wet. Bleach and peroxide can damage skin and discolor clothing. Alcohol tends to be safer on skin and less damaging on skin, especially if misted.

For decontamination purposes, “alcohol” typically refers to two water-soluble chemical compounds—ethyl alcohol and isopropyl alcohol, though it is contemplated that other alcohols could also be used. Alcohols are effective antimicrobial agents as they denature proteins of pathogens. Ethyl alcohol, at concentrations of 60%-80%, is a potent virucidal agent inactivating all of the lipophilic viruses (e.g., herpes, vaccinia, and influenza virus) and many hydrophilic viruses (e.g., adenovirus, enterovirus, rhinovirus, and rotaviruses. Isopropyl alcohol is active against the lipid viruses. The ability of ethyl and isopropyl alcohol to inactivate the hepatitis B virus (HBV) and the herpes virus, and ethyl alcohol to inactivate human immunodeficiency virus (HIV), rotavirus, echovirus, and astrovirus has also been documented.

Isopropyl alcohol, particularly in solutions between 60% and 90% alcohol with 10-40% purified water, has been shown to be rapidly antimicrobial against bacteria, fungi, and viruses. Once alcohol concentrations drop below 50%, isopropyl alcohol's usefulness for disinfection drops sharply. The presence of water has been demonstrated to be important in destroying or inhibiting the growth of pathogenic microorganisms with isopropyl alcohol. Water acts as a catalyst and plays a key role in denaturing the proteins of vegetative cell membranes. 70% solutions penetrate cell walls more completely which permeates the entire cell, coagulates all proteins, thereby causing microorganism death. Extra water content slows evaporation, therefore increasing surface contact time and enhancing effectiveness. Isopropyl alcohol concentrations over 91% coagulate proteins instantly. Consequently, a protective layer is created which protects other proteins from further coagulation.

In this manner, the improved decontamination system of the present invention accomplishes all of the forgoing objectives, thereby providing an easy solution for decontaminating individuals and their clothing when they return home after potential exposure to pathogens. The system provides users with the ability to kill germs, viruses, and bacteria as they pass through the system. The present invention is an apparatus for home or office use that can mist a user with a decontamination solution. Finally, the improved decontamination system of the present invention is capable of improving safety of a user by quickly and easily decontaminating the user so that they do not spread dangerous pathogens throughout their home, thereby reducing the risk of illness.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a decontamination apparatus for disinfecting, sanitizing, or decontaminating a person and clothing. The decontamination apparatus comprises a frame component, a fluid reservoir, a conduit, and a plurality of outlets. The frame component is generally rectangular in shape and defines a decontamination area. The fluid reservoir is configured to retain a decontamination solution. The decontamination solution is typically an alcohol solution capable of being dispensed as a mist.

The plurality of outlets are located around the frame component and are directed generally inward toward the decontamination area. The plurality of outlets are misting nozzles and are fluidly connected to fluid reservoir via the conduit which runs throughout the frame component. The decontamination apparatus further comprises an activation component. The activation component may be manually triggered or triggered by motion to initiate a decontamination cycle. Once the decontamination cycle is initiated, the fluid reservoir releases the decontamination fluid under pressure into the conduit where it is delivered to the plurality of outlets and expelled onto the user as a mist.

In an additional embodiment, a decontamination apparatus comprises a frame component, a fluid reservoir, a conduit, and a rotatable dispensing component. The frame component is generally rectangular in shape and retains the rotatable dispensing component. The rotatable dispensing component is rotatably attached to a top cross pole of the frame component and extends downward. The fluid reservoir is configured to retain a decontamination solution. The decontamination solution is typically an alcohol solution capable of being dispensed as a mist.

The rotatable dispensing component comprises a mist dispensing element and a plurality of outlets. The mist dispensing element is generally curved in shape and rotates within the frame component to define a decontamination area. The plurality of outlets are located along the mist dispensing element and are generally directed inward toward the decontamination area. The plurality of outlets are misting nozzles. The rotatable dispensing component is fluidly connected to fluid reservoir via the conduit which runs throughout the frame component. The decontamination apparatus further comprises an activation component. The activation component may be manually triggered or triggered by motion to initiate a decontamination cycle. Once the decontamination cycle is initiated, the fluid reservoir releases the decontamination fluid under pressure into the conduit where it is delivered to the plurality of outlets and expelled onto the user as a mist.

In an additional embodiment, a decontamination apparatus comprises a frame component, a fluid reservoir, a conduit, and a dispensing component. The frame component is generally rectangular in shape and retains the dispensing component. The dispensing component is horizontally disposed within and vertically repositionable within the frame component. The dispensing component is movably attached to the frame component via a pair of rails along a pair of vertical poles of the frame component via flexible tubing. The decontamination apparatus may further comprise a chamber attached to the frame component. The chamber encapsulates the dispensing component. The fluid reservoir is configured to retain a decontamination solution. The decontamination solution is typically an alcohol solution capable of being dispensed as a mist.

The dispensing component comprises a mist dispensing element and a plurality of outlets. The mist dispensing element is partially circular in shape and defines a decontamination area. The plurality of outlets are located along the mist dispensing element and are directed generally inward toward the decontamination area. The plurality of outlets are misting nozzles. The dispensing component is fluidly connected to fluid reservoir via the conduit which runs throughout the frame component. The decontamination apparatus further comprises an activation component. The activation component may be manually triggered or triggered by motion to initiate a decontamination cycle. Once the decontamination cycle is initiated, the fluid reservoir releases the decontamination fluid under pressure into the conduit where it is delivered to the plurality of outlets and expelled onto the user as a mist.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of a decontamination apparatus of the present invention for misting a person with a decontamination solution in accordance with the disclosed architecture.

FIG. 2 illustrates a perspective view of the decontamination apparatus of the present invention decontaminating the person with the decontamination solution in accordance with the disclosed architecture.

FIG. 3 illustrates a perspective view of an additional embodiment of a decontamination apparatus of the present invention for misting a person with a decontamination solution in accordance with the disclosed architecture.

FIG. 4 illustrates a perspective view of the decontamination apparatus of the present invention decontaminating the person with the decontamination solution in accordance with the disclosed architecture.

FIG. 5 illustrates a perspective view of an additional embodiment of a decontamination apparatus of the present invention for misting a person with a decontamination solution in accordance with the disclosed architecture.

FIG. 6 illustrates a perspective view of the decontamination apparatus of the present invention decontaminating the person with the decontamination solution in accordance with the disclosed architecture.

FIG. 7 illustrates a cutaway perspective view of the decontamination apparatus of the present invention for misting the person with the decontamination solution in accordance with the disclosed architecture.

FIG. 8 illustrates a perspective view of a decontamination chamber of the decontamination apparatus of the present invention for misting the person with the decontamination solution in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, is a portable in-home decontamination station that uses a chamber-style system designed to facilitate decontamination of a person and their clothing upon entering a home. The system is configured to spray approximately between 60-91% alcohol mist onto the user. Motion sensors may be integrated into the system to provide a no-touch ability to decontaminate oneself.

A rectangular structure is configured with several projectiles that spray the mist as the user passes through the structure. Alternatively, a curved spray arm rotates around the user to spray the mist onto the user. Alternatively, the user steps into a partially circular spray arm that moves up and down within the rectangular structure while spraying the mist onto the user. A plastic or nylon chamber may be attached to the rectangular structure to surround the decontamination area to prevent overspray. Overall, the system is intended to sanitize germs, viruses, and bacteria from the user and reduce the chance of spreading illness.

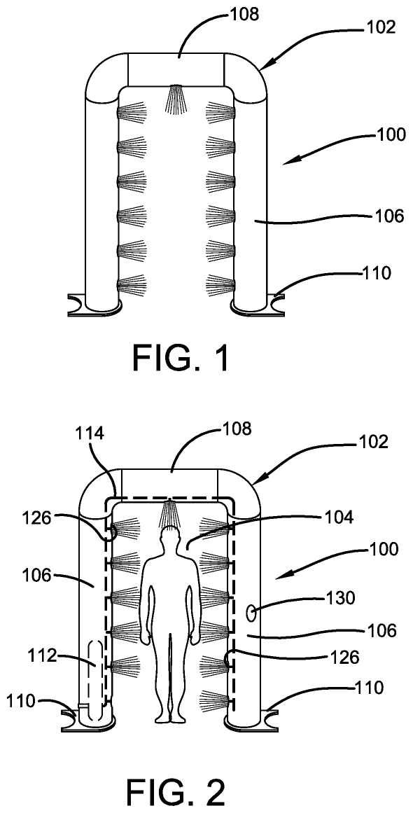

Referring initially to the drawings, FIGS. 1 and 2 illustrate a decontamination apparatus 100. The decontamination apparatus 100 is configured for disinfecting, sanitizing, or decontaminating a person and their clothing. The decontamination apparatus 100 is designed to dispense a mist of a decontaminating alcohol solution. The decontaminating alcohol solution is typically ethyl or isopropyl alcohol in a concentration of approximately between 60-91%, though it is contemplated that other types of alcohol may also be used. This is desirable as this concentration range of alcohol has been shown to be rapidly antimicrobial against bacteria, fungi, and viruses without damaging skin or clothing.

The decontamination apparatus 100 comprises a frame component 102. The frame component 102 is preferably generally rectangular in configuration and is tubular and hollow. The frame component 102 defines a decontamination area 104 where the user can enter and be decontaminated, and is preferably comprised of a plastic, fiberglass, metal or other suitable material. The frame component 102 comprises a pair of vertical poles 106 connected at the top by a top cross pole 108. Each of the pair of vertical poles 106 terminated at the bottom in a base support 110 for stability. The frame component 102 is dimensioned to receive a human being within.

The decontamination apparatus 100 further comprises a fluid reservoir 112, a conduit 114, and a plurality of outlets 126. The fluid reservoir 112 is configured to retain the decontamination solution and would have a closeable opening therein for receiving the solution. The fluid reservoir 112 is constructed to retain the decontamination solution under pressure (e.g., a pressurized gas cartridge or pump and associated power supply). The conduit 114 is a tube or hose in fluid communication with and connected to the fluid reservoir 112.

The plurality of outlets 126 are located around the frame component 102 and are directed generally inward toward and cover the defined decontamination area 104. The plurality of outlets 126 are misting nozzles and are fluidly connected to fluid reservoir 112 via the conduit 114 which runs throughout the frame component 102. The decontamination apparatus 100 further comprises an activation component 130. The activation component 130 may be manually triggered or triggered by motion, such as by a motion activated trigger, sensor, button or switch to initiate a decontamination cycle. Once the decontamination cycle is initiated, the fluid reservoir 112 releases the decontamination fluid under pressure into the conduit 114 where it is delivered to the plurality of outlets 126 and expelled onto the user as an alcohol mist as illustrated in FIG. 2. Experimental testing has shown that an ideal decontamination cycle will last between four and fifteen seconds. Once the decontamination cycle is complete, the user exits the decontamination apparatus 100 and allows the alcohol mist to dry.

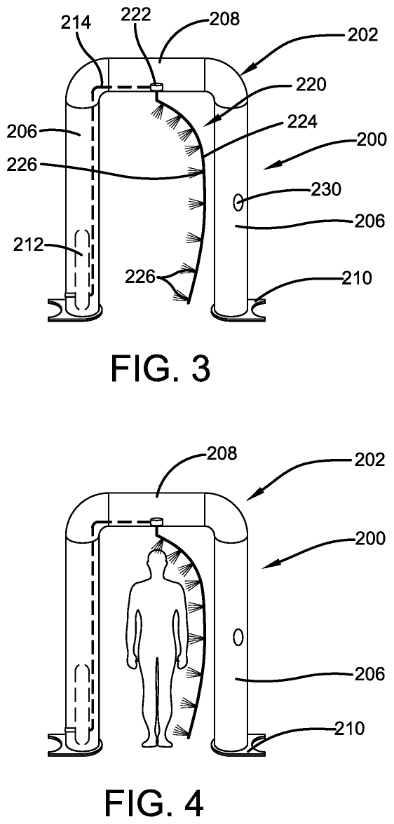

In an additional embodiment as illustrated in FIGS. 3 and 4, a decontamination apparatus 200 comprises a frame component 202, a fluid reservoir 212, a conduit 214, and a rotatable dispensing component 220. The frame component 202 is generally rectangular in shape and retains the rotatable dispensing component 220. The frame component 202 is preferably generally rectangular in configuration and is tubular and hollow. The frame component 202 defines a decontamination area 204 where the user can enter and be decontaminated. The frame component 202 comprises a pair of vertical poles 206 connected at the top by a top cross pole 208. Each of the pair of vertical poles 206 terminated at the bottom in a base support 210 for stability. The frame component 202 is dimensioned to receive a human being within.

The fluid reservoir 212 is constructed to retain the decontamination solution under pressure. The conduit 114 is a tube or hose in fluid communication with and connected to the fluid reservoir 112. The decontamination is typically an alcohol solution capable of being dispensed as a mist as discussed supra.

The rotatable dispensing component 220 is vertically disposed and rotatably attached to the top cross pole 208 of the frame component 202 extending downward. The rotatable dispensing component 220 comprises a mist dispensing element 224 and a plurality of outlets 226. The mist dispensing element 224 is generally curved in shape and rotates within the frame component 202 to define a decontamination area. The rotatable dispensing component 220 further comprises a conduit attachment element 222 rotatably connecting the mist dispensing element 224 to the conduit 214.

The plurality of outlets 226 are located along the mist dispensing element 224 and are generally directed inward toward the decontamination area. The plurality of outlets 226 are typically misting nozzles. The rotatable dispensing component 220 is fluidly connected to fluid reservoir 212 via the conduit 214 which runs throughout the frame component 202. The decontamination apparatus 200 further comprises an activation component 230. The activation component 230 may be manually triggered or triggered by motion, such as by a motion activated trigger, to initiate a decontamination cycle. Once the decontamination cycle is initiated, the fluid reservoir 212 releases the decontamination fluid under pressure into the conduit 214 where it is delivered to the plurality of outlets 226 and expelled onto the user as a mist as the mist dispensing element 224 rotates around the user to provide full coverage as illustrated in FIG. 4. Once the decontamination cycle is complete, the user exits the decontamination apparatus 200 and allows the alcohol mist to dry.

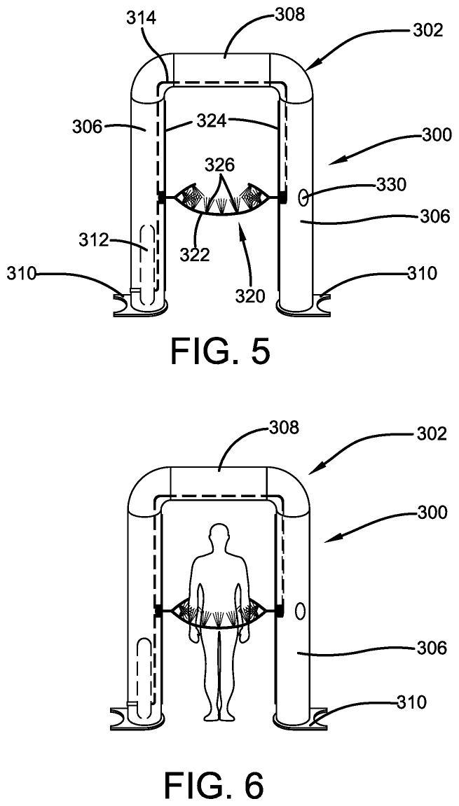

In an additional embodiment as illustrated in FIGS. 5-8, a decontamination apparatus 300 comprises a frame component 302, a fluid reservoir 312, a conduit 314, and a dispensing component 320. The frame component 302 is generally rectangular in shape and retains the dispensing component 320. The frame component 302 is generally rectangular in configuration and is tubular and hollow. The frame component 302 defines a decontamination area 304 where the user can enter and be decontaminated. The frame component 302 comprises a pair of vertical poles 306 connected at the top by a top cross pole 308. Each of the pair of vertical poles 306 terminated at the bottom in a base support 310 for stability. The frame component 302 is dimensioned to receive a human being within.

As illustrated in FIG. 8, the decontamination apparatus 300 may further comprise a chamber 340. The chamber 340 is attached to the frame component 302 and encapsulates the dispensing element 320. The chamber 340 is typically a plastic or nylon chamber with a closable opening 342, such as a zipper. The chamber 340 effectively retains any potential overspray that could occur during a decontamination cycle, but is ventilated so that a user can breathe during the decontamination cycle. In an alternative embodiment, the chamber 340 does not have to be completely encompassing and may be a partial chamber 340.

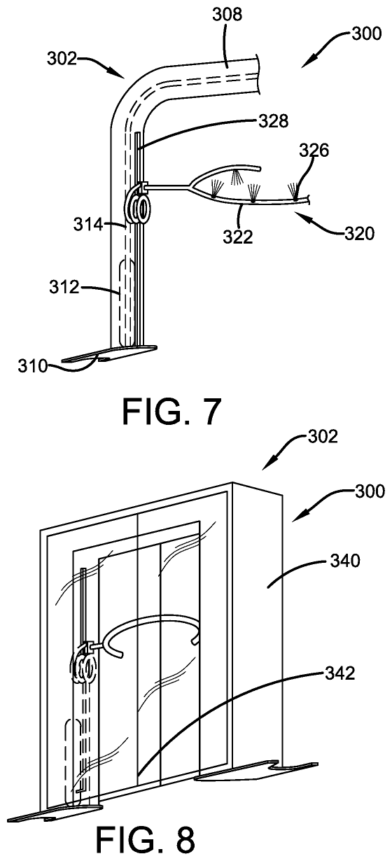

As illustrated in FIGS. 5 and 7, the dispensing component 320 is horizontally disposed within and vertically movable within the frame component 302. The dispensing component 320 is movable attached to the frame component 302 via a pair of rails 328 along the pair of vertical poles 306 of the frame component 302. The fluid reservoir 312 is configured to retain a decontamination solution under pressure. The conduit 314 is a tube or hose in fluid communication with and connected to the fluid reservoir 312. The decontamination solution is typically an alcohol solution capable of being dispensed as a mist as described supra.

The dispensing component 320 comprises a mist dispensing element 322 and a plurality of outlets 326. The mist dispensing element 322 is partially circular in shape and partially circles a decontamination area. The plurality of outlets 326 are located along the mist dispensing element 322 and are directed inward toward the decontamination area. The plurality of outlets 326 are misting nozzles. The dispensing component 320 is fluidly connected to fluid reservoir 312 via the conduit 314 which runs throughout the frame component 302. The decontamination apparatus 300 further comprises an activation component 330. The activation component 330 may be manually triggered or triggered by motion, such as by a motion activated trigger, to initiate a decontamination cycle. Once the decontamination cycle is initiated, the fluid reservoir 312 releases the decontamination fluid under pressure into the conduit 314 where it is delivered to the plurality of outlets 326 and expelled onto the user as a mist as the dispensing component 320 moves up and down within the frame component 302 to provide full coverage as illustrated in FIG. 6. Once the decontamination cycle is complete, the user exits the decontamination apparatus 300 and allows the alcohol mist to dry.

It is contemplated that the decontamination apparatus 100, 200, and 300 constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

Notwithstanding the forgoing, the decontamination apparatus 100, 200, and 300 of the present invention and its various structural components can be of any suitable size, shape, and configuration as is known in the art without affecting the overall concept of the invention, provided that it accomplishes the above stated objectives. One of ordinary skill in the art will appreciate that the shape and size of the decontamination apparatus 100, 200, and 300 and its various components and material, as shown in the FIGS. are for illustrative purposes only, and that many other shapes and sizes of the decontamination apparatus 100, 200, and 300 are well within the scope of the present disclosure. Although the dimensions of the decontamination apparatus 100, 200, and 300 are important design parameters for attaining decontamination, the decontamination apparatus 100, 200, and 300 and its components may be of any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A decontamination apparatus comprising:
a frame component defining a decontamination area;
a fluid reservoir for retaining a decontamination solution;
a conduit in fluid communication with the fluid reservoir; and
a plurality of outlets positioned around the frame component in fluid communication with the conduit; and
wherein the decontamination solution is expelled via the plurality of outlets.

2. The decontamination apparatus of claim 1, wherein the decontamination solution is an alcohol solution of between 60% and 90% ethyl or isopropyl alcohol.

3. The decontamination apparatus of claim 1, wherein the plurality of outlets are misting nozzles.

4. The decontamination apparatus of claim 1, wherein the plurality of outlets are positioned to cover the decontamination area.

5. The decontamination apparatus of claim 1, wherein the fluid reservoir is constructed to retain the decontamination solution under pressure.

6. The decontamination apparatus of claim 1 further comprising an activation component for initiating a decontamination cycle.

7. The decontamination apparatus of claim 6, wherein the activation component is motion activated.

8. A decontamination apparatus comprising
a frame component;
a fluid reservoir for retaining a decontamination solution;
a conduit in fluid communication with the fluid reservoir; and
a rotatable dispensing component comprising a mist dispensing element and a plurality of outlets in fluid communication with the conduit; and
wherein the decontamination solution is expelled via the plurality of outlets.

9. The decontamination apparatus of claim 8, wherein the plurality of outlets are misting nozzles.

10. The decontamination apparatus of claim 8, wherein the fluid reservoir is pressurized.

11. The decontamination apparatus of claim 8, wherein the mist dispensing element rotates to define a decontamination area within the frame component.

12. The decontamination apparatus of claim 8, wherein the rotatable dispensing component further comprises a conduit attachment element rotatably connecting the mist dispensing element to the conduit.

13. The decontamination apparatus of claim 8, wherein the dispensing component extends downward from a top cross pole of the frame component.

14. The decontamination apparatus of claim 8 further comprising an activation component for initiating a decontamination cycle.

15. A decontamination apparatus comprising:
a frame component;
a fluid reservoir for retaining a decontamination solution;
a conduit in fluid communication with the fluid reservoir; and
a dispensing component comprising a mist dispensing element and a plurality of outlets in fluid communication with the conduit; and
wherein the decontamination solution is expelled via the plurality of outlets.

16. The decontamination apparatus of claim 15, wherein the mist dispensing element partially circles a decontamination area.

17. The decontamination apparatus of claim 15, wherein the dispensing component is movable vertically within the frame component.

18. The decontamination apparatus of claim 15, wherein the dispensing component is movably attached to the frame component via a pair of rails.

19. The decontamination apparatus of claim 15 further comprising a decontamination chamber attached to the frame component.

20. The decontamination apparatus of claim 15 further comprising an activation component for initiating a decontamination cycle.
